SEQUENCE LISTING (4 sequences)

SEQ ID NO: 1 - protein, 70 aa, Artificial Sequence
  SEQ ID NO 1 is the sequence of native PF-4 protein.
EAEEDGDLQCLCVKTTSQVRPRHITSLEVIKAGPHCPTAQLIATLKNGRKICLDLQAPLYKKIIKKLLES

SEQ ID NO: 2 - protein, 70 aa, Artificial Sequence
  SEQ ID NO. 2 is the mutated sequence of PF-4 (Arg in position 56)
EAEEDGDLQCLCVKTTSQVRPRHITSLEVIKAGPHCPTAQLIATLKNGRKICLDLRAPLYKKIIKKLLES

SEQ ID NO: 3 - protein, 24 aa, Artificial Sequence
  SEQ ID NO. 3 is the 47-70 fragment of mutated PF-4 SEQ ID. NO. 2
NGRKICLDLRAPLYKKIIKKLLES

SEQ ID NO: 4 - protein, 42 aa, Artificial Sequence
  SEQ ID NO. 4 is the 17-34/47-70 fragment of mutated PF-4 SEQ. ID NO. 2.
SQVRPRHITSLEVIKAGPNGRKICLDLRAPLYKKIIKKLLES

(19) United States
(12) Patent Application Publication
(10) Pub. No.: US 2004/0086999 A1
(43) Pub. Date: May 6, 2004

(54) Mutated-pf-4, its fragments and mutated fusion peptides, their analogues, corresponding dna, cdna and mrna sequences and their use for inhibiting angiognesis

(76) Inventors: Andreas Bikfalvi, Gradignan (FR); Monica Alemany, Paris (FR)

Correspondence Address: Piper Rudnick, P O Box 64087, Chicago, IL, 60664-0807, US

(21) Appl. No.: 10/333,364

(22) PCT Filed: Jul. 18, 2001
(86) PCT No.: PCT/FR01/02341

(30) Foreign Application Priority Data
Jul. 19, 2000 (FR) 00/09464

(51) Int. Cl. C07H 21/04; C12P 21/04; C12N 9/64

(52) U.S. Cl. 435/226; 536/23.2; 435/69.7; 435/320.1; 435/325

(57) ABSTRACT

The invention concerns a peptide selected among the group consisting of PF-4, fragments and fusion peptides derived from PF-4, and their analogues, having an angiogenesis-inhibiting activity, wherein the glutamine in position 56 in the native PF-4 is replaced by an arginine, a lysine or a histidine, preferably an arginine. The inventive peptide has an I₅₀ less than the I₅₀ of the same peptide not having a mutation in 56. The invention also concerns DNA or cDNA sequences coding for said peptides and the use of said sequences and/or said peptides for preparing a medicine inhibiting angiogenesis and/or proliferation of cells.

The present invention concerns a peptide corresponding to the mutated platelet factor 4 (PF-4), or to its mutated fusion peptides and fragments, or to their analogues. It also concerns the corresponding DNA and cDNA and mRNA sequences, and the use of these peptides and sequences for inhibiting angiogenesis. More particularly, the invention concerns a PF-4 carrying a mutation in position 56, the glutamine (Q) being replaced by a basic amino acid such as arginine (R), lysine (K) or histidine (H), or a fragment or fusion peptide derived from PF-4 and comprising this mutation, or their analogues.

PF-4 is a known protein whose 70 amino acids (sequence 1-70, see SEQ. ID. No:1) have been sequenced.

It has been shown that native PF-4 has some inhibitory activity in vitro and in vivo on angiogenesis and the proliferation of endothelial cells, and can therefore be used in the treatment of angiogenesis and consequently of pathologies which are related thereto, such as for example cancers, tumors and chronic inflammatory diseases.

The mode of action of PF-4 is still not completely known. However, it has been shown that it inhibits the binding of FGF-2 (Fibroblast Growth Factor) and VEGF (Vascular Endothelial Growth Factor) to their receptors, and the binding of FGF-2 to proteoglycans. PF-4 also inhibits the dimerization of FGF-2.

It is sought, however, to increase the efficacy of the treatments of angiogenesis, and in particular to increase the activity of the inhibitors, in order to be able either to limit the quantity of inhibitor used during the treatment while keeping the inhibitory activity constant, or to obtain a higher inhibitory activity with the same quantity of inhibitor.

It has therefore been sought to obtain, from PF-4, peptides having a higher activity than that of native PF-4. Several methods have been used.

One research route has consisted in conjugating a PF-4 (or a fragment or mutant thereof) with another entity which makes it possible to increase the inhibitory activity of PF-4.

In particular, EP patent 723 015 describes the use of a polypeptide conjugate comprising PF-4 or a fragment or mutant thereof, conjugated with a second molecule chosen from fluorophores, chelators, carrier molecules, antibodies, toxins and cell receptor molecules or their complementary ligands. In this same document, it is specified that a peptide of 13 amino acids corresponding to the C-terminal sequence of PF-4 in which the two pairs of lysine residues have been replaced by two glutamic acid-glutamine pairs has an angiogenesis-inhibiting activity.

Patent applications WO 93/02192 and WO 95/12414 also describe the conjugation of PF-4 and of another entity.

Another research route has consisted in deriving, from native PF-4, polypeptides which are more active than this PF-4.

Patent application EP 378 364 describes peptides and fragments derived from PF-4. In particular, it describes a protein of about 71 amino acids comprising amino acids 1 to 60 of native PF-4, it being possible for amino acids 61 and those that follow (C-terminal end) to correspond to different sequences, The importance of the inhibitory activity of the sequence of 13 amino acids corresponding to the C-terminal end of native PF-4 is particularly emphasized.

Patent application EP 176 588 relates to different polypeptides of 15 to 80 amino acids having a number of mutations compared with the PF-4 sequence. A great number of sequences are covered by the formulae given, all these sequences having an acid N-terminal end (that is to say presence of glutamic or aspartic acid among the 6 to 15 N-terminal amino acids), and a basic C-terminal end (that is to say presence of lysine or arginine among the 6 to 15 C-terminal amino acids).

Unlike the teaching of the prior art, which involves conjugation with a second molecule or numerous mutations compared with the sequence of the native PF-4, the applicants have observed, surprisingly, that a single mutation at the level of amino acid 56 of the PF-4 sequence, or of an analogue or fragment or fusion peptide derived from PF-4 and comprising this amino acid, makes it possible to considerably increase the angiogenesis-inhibiting activity of the peptide obtained.

The invention therefore concerns a PF-4 or its analogues in which the glutamine (Q) in position 56 is replaced by a basic amino acid. This basic amino acid may be arginine (R) (see SEQ. ID, No:2), lysine (K) or histidine (H). Arginine is preferred. The invention also concerns a fragment or a fusion peptide derived from PF-4, or one of their analogues, comprising this mutation.

The term “PF-4 peptide according to the invention” will designate all these peptides, namely PF-4, its analogues, the fragments and fusion peptides derived from PF-4 and their analogues, comprising this particular mutation at the level of the amino acid corresponding to glutamine 56 in the sequence of native PF-4.

A nonmutated PF-4 peptide will be referred to as “DLQ”. A PF-4 peptide comprising the mutation in position 56, that is to say a PF-4 peptide according to the invention, will be referred to as “DLR”.

The term “analogues” denotes peptides whose sequences exhibit, in relation to each other, mutations which have no harmful consequence on the activity of the peptide. In the present application, the analogues will all have an angiogenesis-inhibiting activity. The mutations may consist in particular in the replacement of an amino acid by an amino acid of the same type, such as for example the replacement of a valine (V) by an alanine (A), which are both aliphatic amino acids, or the replacement of a threonine (T) by a serine (S), which are both oxy-substituted aliphatic amino acids.

A mutated fragment according to the invention may correspond in particular to the mutated fragment 47-70 (see SEQ. ID. No:3).

Any fragment derived from native PF-4 having angiogenesis-inhibiting activity also forms part of the fragments according to the invention, whatever its length, provided that it comprises this particular mutation at a level of the amino acid corresponding to glutamine 56 in the sequence of native PF-4. This may involve a fragment having, compared with PF-4, fewer amino acids and/or additional amino acids (an example is fragment 47-70).

A mutated fusion peptide according to the invention may correspond in particular to the fusion peptide 17-34/47-70 (see SEQ. ID. No:4). Tests have shown that this fusion peptide has an inhibitory activity greater than that of native PF-4 or of fragment 47-70. Such a mutated fusion peptide will therefore be particularly advantageous.

Any fusion peptide derived from native PF-4 having angiogenesis-inhibiting activity also forms part of the fusion peptides according to the invention, provided that it comprises this particular mutation at the level of the amino acid corresponding to glutamine 56 in the sequence of native PF-4.

The number 56 is given to the mutated amino acid with reference to its position in the sequence of native PF-4. It is evident that this amino acid will not necessarily be at the same position 56 in all the PF-4 peptides according to the invention. For example, in fragment 47-70, the amino acid carrying the mutation in 56 is in fact at position 10. The term “mutation in 56” will nevertheless be used regardless of the real position of the amino acid in the sequence of the peptide.

A PF-4 peptide according to the invention will have an I₅₀ (peptide concentration which makes it possible to obtain 50% inhibition) less than the I₅₀ value for the same peptide not mutated in position 56.

Preferably, the I₅₀ of a PF-4 peptide according to the invention will be 2 to 20 less, preferably 4 to 15-fold less, still more preferably 5 to 10-fold less than the I₅₀ value for the same peptide not having the mutation, that is to say for the peptide having an identical sequence except for the mutation in position 56.

The I₅₀ can be measured by conventional techniques known to persons skilled in the art, such as that described in Jouan V., Canron X., Alemany M., Caen J., Quentin G., Plouet J., and Bikfalvi A. (1999), Modulation of in vitro angiogenesis by Platelet factor-4 derived peptides and mechanism of action, Blood, 94: 984-93, or in Perollet C., Han Z. C., Savona C., Caen J. P., and Bikfalvi A. (1998) Platelet Factor-4 modulates Fibroblast Growth Factor-2 Activity and inhibits FGF-2 dimerization, Blood 91 : 1-12.

The PF-4 peptides according to the invention may be obtained by the usual techniques known to persons skilled in the art, namely in particular from native PF-4 (enzymatic cleavage), by synthesis, by recombinant techniques, by combinatorial chemistry.

The invention also concerns a pharmaceutical composition comprising one of the PF-4 peptides according to the invention, in combination with one or more pharmaceutically acceptable excipients. This composition may be used for the treatment or prevention of angiogenesis-related diseases, such as cancer, vascular diseases of the retina (retinopathies), chronic inflammatory diseases (such as chronic rheumatoid arthritis), angiomas, hemangiomas and certain hemopathies (leukemias). It may be administered, for example, intravenously or subcutaneously.

The invention also concerns the DNA, cDNA or mRNA sequences coding for the PF-4 peptides according to the invention, which sequences are obtained according to techniques known to persons skilled in the art.

The invention also concerns the use of the PF-4 peptides according to the invention and/or of the DNA, cDNA or mRNA sequences for the preparation of a medicine for the treatment or prevention of angiogenesis-related diseases. Indeed, taking into account the results obtained in vitro, it is expected that angiogenesis in vivo (for example on rabbit cornea) and angiogenesis ex vivo (for example inhibition of angiogenesis on the chorioallantoic membrane) will also be inhibited. Such a medicine may be administered, for example, intravenously or subcutaneously.

### EXAMPLES

Tests were carried out in order to evaluate the inhibitory activity of a mutated peptide 47-70 compared with the control peptide 47-70. A mutation in position 56 corresponds here to arginine.

More particularly, the inhibitory effect of each peptide was evaluated by testing the inhibitory effect on the binding of FGF-2 or VEGF to their high-affinity receptors (Table 1), the binding of FGF-2 to proteoglycans (Table 2), and cell proliferation (Table 3).

The inhibition of the binding of FGF-2 to high-affinity receptors (Table 1) is measured in the following manner. FGF-2 is radiolabeled with ¹²⁵I-Na, and 10 ng/ml are incubated with endothelial cells for 2 h at 4° C. The radioactivity bound to high-affinity receptors is then determined. The specific binding is estimated by subtracting the nonspecific binding from the total binding (in the presence of a 100-fold excess of cold ligand relative to the hot ligand). The I₅₀ is the concentration which inhibits specific binding by 50%.

The inhibition of the binding of VEGF to the surface receptors (Table 1) is measured in the following manner. VEGF is radiolabeled with ¹²⁵I-Na, and 10 ng/ml are incubated with endothelial cells for 2 h at 4° C. The radioactivity bound to the receptors is then determined. The specific binding is estimated by subtracting the nonspecific binding from the total binding (in the presence of a 100-fold excess of cold ligand relative to the hot ligand). The I₅₀ is the concentration which inhibits specific binding by 50%.

The inhibition of the binding of FGF-2 to the proteoglycans (Table 2) is measured in the following manner. FGF-2 is radiolabeled with ¹²⁵I-Na, and 10 ng/ml are incubated with endothelial cells for 2 h at 4° C. The radioactivity bound to the proteoglycans is then determined. The specific binding is estimated by subtracting the nonspecific binding from the total binding (in the presence of a 100-fold excess of cold mutant relative to the hot ligand). The I₅₀ is the concentration which inhibits specific binding by 50%.

The inhibition of the biological activity of FGF-2 (Table 3) is measured in the following manner: 20 000 cells are incubated with 10 ng/ml of FGF-2 in DMEM medium containing 1% fetal calf serum (FCS) in dishes 3.5 cm in diameter in the presence or in the absence of peptide. The cells are counted on the 6 day using a Coulter counter. The experiments are carried out in duplicate. The I₅₀ is the concentration which inhibits the proliferation induced by FGF-2 by 50%.

A more detailed description of these measurements of I₅₀ may be found in Jouan et al. and in Perollet et al. cited above.

The activities of the peptide 47-70 DLR, that is to say the peptide mutated in position 56, are compared with that of the nonmutated peptide 47-70, by way of a comparative example.

### TABLE 1

I₅₀ values corresponding to the inhibition of the binding of FGF-2 or of VEGF to the high-affinity receptors by each peptide:

TABLE 2

| | Binding of | Binding of |
|---|---|---|
| | FGF-2 | VEGF |
| Peptide 47-70 DLQ | 1.5 μM | 1.5 μM |
| Peptide 47-70 DLR | 0.3 μM | 0.3 μM |

I₅₀ values corresponding to the inhibition of the binding of FGF-2 to proteoglycans by each peptide:

TABLE 3

| | |
|---|---|
| Peptide 47-70 DLQ | 3.5 μM |
| Peptide 47-70 DLR | 0.6 μM |

I₅₀ values corresponding to the inhibition of cell proliferation by each peptide:

| | |
|---|---|
| Peptide 47-70 DLQ | 2.8 μM |
| Peptide 47-70 DLR | 0.4 μM |

It can be observed, in each case, that the mutated peptide has I₅₀ values which are far lower less than the I₅₀ values for the control peptide. In other words, in these tests, the same inhibition is obtained with a concentration of peptide according to the invention 5 to 7 times less than the concentration of nonmutated peptide.

Such results therefore make it possible to obtain an inhibition 5 to 7 times higher with the same peptide concentration, or on the contrary to obtain the same inhibition but with a peptide concentration 5 to 7 times lower.

This illustrates the considerably higher inhibitory activity of the PF-4 peptides according to the invention.

1. A peptide chosen from the group comprising PF-4, fragments and fusion peptides derived from PF-4, and their analogues, having an angiogenesis-inhibiting activity, in which the glutamine situated in position 56 in the native PF-4 is replaced by a basic amino acid.

2. The peptide as claimed in claim 1, in which the basic amino acid is arginine, lysine or histidine, preferably arginine.

3. The peptide as claimed in either of claims 1 and 2, corresponding to fragment 47-70 of the native PF-4.

4. The peptide as claimed in either of claims 1 and 2, corresponding to the fusion peptide 17-34/47-70.

5. The peptide as claimed in any one of claims 1 to 4, characterized in that it has an I₅₀ less than the I₅₀ of the same peptide not having the mutation in 56.

6. The peptide as claimed in claim 5, characterized in that it has an I₅₀ 2 to 20 less, preferably 4 to 15 times less, still more preferably 5 to 10 times less than the I₅₀ of the same peptide not having the mutation in 56.

7. The peptide as claimed in any one of claims 1 to 6, characterized in that it is obtained by enzymatic cleavage of a native PF-4, by chemical synthesis, by recombinant techniques, or by combinatorial chemistry.

8. A DNA or cDNA or mRNA sequence coding for a peptide as claimed in any one of claims 1 to 7.

9. A medicine comprising a peptide as claimed in any one of claims 1 to 7.

10. A medicine as claimed in claim 9, for the treatment or prevention of angiogenesis-related diseases.

11. The use of a peptide as claimed in any one of claims 1 to 7 or of a DNA or cDNA or mRNA sequence as claimed in claim 8 for the preparation of a medicine for the treatment or prevention of angiogenesis-related diseases, such as cancer, vascular diseases of the retina (retinopathies), chronic inflammatory diseases (such as chronic rheumatoid arthritis), angiomas, hemangiomas and certain hemopathies (leukemias).
